(19) United States
(12) Patent Application Publication
(10) Pub. No.: US 2026/0157834 A1
(43) Pub. Date: Jun. 11, 2026

(54) RETRACTABLE ELECTRIC TOOTHBRUSH

(71) Applicant: Guangdong huale science and technology development co., ltd, Guangzhou (CN)

(72) Inventors: Xin Wang, Guangzhou (CN); Ruidong Li, Guangzhou (CN)

(21) Appl. No.: 19/182,218

(22) Filed: Apr. 17, 2025

Related U.S. Application Data
(63) Continuation-in-part of application No. 17/598,234, filed on Sep. 24, 2021, now Pat. No. 12,303,016.

(30) Foreign Application Priority Data
Apr. 10, 2025 (CN) 202520678677.4

(51) Int. Cl. A61C 17/22; A46B 5/00; A46B 13/02; A61L 2/10

(52) U.S. Cl. CPC A61C 17/225; A46B 5/005; A46B 5/0095; A46B 13/02; A61C 17/222; A61L 2/10

(57) ABSTRACT

A retractable electric toothbrush comprises a handle and a toothbrush head; a mounting plate is fixedly installed inside the handle; one side of the mounting plate is slidably connected to a mounting cylinder, and a driving assembly is arranged between the mounting cylinder and the mounting plate; the driving assembly is configured to drive the mounting cylinder to move vertically inside the handle, thereby causing the toothbrush head to extend or retract into the handle; a control mainboard is installed on an other side of the mounting plate, and a waterproof cover plate is connected to an outer side adjacent to the control mainboard; a sealing gasket is connected between the waterproof cover plate and the mounting plate. The invention achieves rapid movement of the toothbrush head during use through the mounting plate and driving assembly installed inside the handle, enabling quick retractable adjustment.

### TECHNICAL FIELD

The invention relates to the technical field of remote electric toothbrushes, in particular to a retractable electric toothbrush.

### BACKGROUND ART

Electric toothbrush is a kind of oral care tool that drives the toothbrush head through an electric motor core to perform high-frequency vibration or rotation, thus realizing the function of cleaning teeth. It generally consists of a handle, battery, an electric motor, a toothbrush head and a control system. Compared with traditional manual toothbrushes, electric toothbrushes can more efficiently remove dental plaque and food debris, while its vibration frequency and strength is relatively stable, which can reduce damages to the teeth and gums caused by improper brushing strength. In addition, some electric toothbrushes also have multiple cleaning modes, such as daily cleaning, sensitive care, whitening, etc., to meet the oral care needs of different users.

When existing electric toothbrushes are in use, they are usually carried with toothtoothbrush heads detachably mounted on the handles in the travel or going out, and are prone to cause bending at the brush-handle junction, potentially leading to structural damage. Additionally, the exposed toothbrush head design increases contamination risks during storage and transit. Therefore, in view of this, a retractable electric toothbrush is provided after study and improvement based on the current structure and shortcomings, in order to achieve purpose of more practical value.

### SUMMARY OF THE INVENTION

In order to solve problems mentioned above, the invention provides a retractable electric toothbrush, reached by the following technical means:
a retractable electric toothbrush, comprising a handle and a toothbrush head; a mounting plate is fixedly installed inside the handle; one side of the mounting plate is slidably connected to a mounting cylinder, and a driving assembly is arranged between the mounting cylinder and the mounting plate; the driving assembly is configured to drive the mounting cylinder to move vertically inside the handle, thereby causing the toothbrush head to extend or retract into the handle; a control mainboard is installed on an other side of the mounting plate, and a waterproof cover plate is connected to an outer side adjacent to the control mainboard; a sealing gasket is connected between the waterproof cover plate and the mounting plate.

Further, an electric motor core for driving the toothbrush head is installed at a top end of the mounting cylinder, and the toothbrush head is detachably connected to a driving end of the electric motor core.

Further, the driving assembly comprises a rack fixedly installed on one side of the mounting plate and a drive motor fixedly installed inside the mounting cylinder; an output end of the drive motor is transmission-connected to a speed reducer; a drive shaft is provided on one side of the speed reducer, and a gear is fixedly sleeved at an end of the drive shaft, the gear meshes with the rack.

Further, a sealing ring is fixedly sleeved on an outer side of the speed reducer, and the sealing ring is sealingly connected to an inner wall of the mounting cylinder; a locking base is connected to a bottom end of the speed reducer, and the locking base is tightly clamped to a bottom end of the mounting cylinder.

Further, both left and right ends on one side of the mounting plate are provided with limit sliding grooves, and both left and right ends on one side of the mounting cylinder are provided with limit strips; the limit strips are slidably installed in the corresponding limit sliding grooves.

Further, a battery, a disinfection component, and a Hall element are fixedly installed on the control mainboard. When the toothbrush head is fully retracted inward, the disinfection component is provided corresponding to the toothbrush head; a through slot is provided on the mounting plate near the installation position of the disinfection component, and an end of the disinfection component extends through the through slot; waterproof sealant is filled between the through slot and the end of the disinfection component; the disinfection component is an ultraviolet disinfection lamp.

Further, a magnet is fixedly embedded on one side of the mounting cylinder; when the toothbrush head is fully extended outward, the magnet is provided corresponding to the Hall element.

Further, an upper cover and a lower cover are fixedly installed at upper and lower ends of the handle, respectively; a center of the upper cover is provided with a through hole for the toothbrush head to extend out; the lower cover is provided with a plurality of drainage holes and a charging port; a waterproof cover is connected to the charging port, and a charging connector is fixedly installed inside the charging port, with the charging connector electrically connected to the control mainboard.

Further, two baffles are installed on a bottom of the upper cover to seal the through hole of the upper cover; a fixed disk is fixedly installed at a top end of the mounting plate, and the fixed disk is provided with a through slot corresponding to the through hole; two sets of connection holes are provided at one end of the fixed disk near the through slot; one end of each baffle is provided with a mounting shaft, and the mounting shafts are rotatably installed in the corresponding connection holes; fixed shafts are provided on both sides of the fixed disk near the through slot, and connection shafts corresponding to the fixed shafts are provided on one side of each baffle; springs are connected between the corresponding connection shafts and the fixed shafts.

Further, one end of each baffle near the mounting shaft is shaped as an arc, and arc surfaces are provided with gear slots; the gear slots of two baffles mesh with each other to ensure synchronous movement of two baffles, achieving synchronized opening and closing; a bottom side of each baffle is provided with an inclined surface; when the toothbrush head extends outward, its end drives the baffles to compress the springs by pressing against inclined surfaces, thereby completing the opening of the baffles.

Compared with the prior art, the invention has the following advantageous effects:
1. the invention achieves rapid movement of the toothbrush head during use through the mounting plate and driving assembly installed inside the handle, enabling quick retractable adjustment, which facilitates swift storage and protection of the toothbrush head during use or transportation, preventing contamination or damage. Additionally, the disinfection component installed inside the handle provides rapid disinfection when the toothbrush head is retracted, ensuring readiness for subsequent use;
2. by providing a waterproof cover plate and a sealing ring one one side of the mounting plate, the invention ensures waterproof performance of an electrical-control component on one side of the mounting plate during use, thereby guaranteeing long-term stable operation; meanwhile, a sealing ring is mounted between the speed reducer and the mounting cylinder, which ensures a well-sealed space for the drive motor to prevent water ingress, thereby guaranteeing overall waterproof performance.

### BRIEF DESCRIPTION OF ACCOMPANY DRAWINGS

FIG. 1 is a first schematic diagram of the toothbrush head fully retracted in the invention;

FIG. 2 is a second schematic diagram of the toothbrush head fully retracted in the invention;

FIG. 3 is a schematic diagram of the toothbrush head fully extended in the invention;

FIG. 4 is an exploded view of the structure of the invention;

FIG. 5 is a first exploded view of components on both sides of the mounting plate in the invention;

FIG. 6 is a second exploded view of components on both sides of the mounting plate in the invention;

FIG. 7 is an exploded view of the mounting cylinder in the invention;

FIG. 8 is an exploded view of the fixed disk and baffles in the invention;

FIG. 9 is a schematic diagram of the baffles in the invention.

In the drawings, the corresponding relationship between the component names and the reference numbers is as follows:

1 handle, 2 upper cover, 3 lower cover, 4 toothbrush head, 5 mounting plate, 6 mounting cylinder, 7 control mainboard, 8 battery, 9 sealing gasket, 10 waterproof cover plate, 11 disinfection component, 12 Hall element, 13 rack, 14 magnet, 15 drive motor, 16 speed reducer, 17 sealing ring, 18 gear, 19 locking base, 20 fixed disk, 21 baffle, 22 connection shaft, 23 mounting shaft.

### SPECIFIC EMBODIMENT OF THE INVENTION

The following embodiments of the invention are described in further detail in connection with the accompanying drawings and embodiments. The following embodiments are used to illustrate the invention, but cannot be used to limit the scope of the invention.

In the description of the invention, unless otherwise indicated, “plurality” means two or more; the terms “top”, “bottom”, “left”, “right”, “inner”, “outer”, “front end”, “rear end”, “head”, “tail”, and the like indicate orientation or positional relationships based on those shown in the accompanying drawings, solely for the purpose of facilitating the description of the invention and simplifying the description, it is not intended to indicate or imply that the device or element referred to must have a particular orientation, be constructed and operated in a particular orientation, and therefore cannot be construed as a limitation of the present invention. Furthermore, the terms “first”, “second”, “third”, etc. are used for descriptive purposes only and are not to be understood as indicating or implying relative importance.

In the description of the present invention, it is to be noted that, unless otherwise expressly provided and limited, the terms “connected”, “linked” are to be understood in a broad sense, for example, it can be fixed connection, removable connection, or integrally connected; it may be a mechanical connection or an electrical connection; it may be a direct connection or an indirect connection through an intermediate medium. For those of ordinary skill in the art, the specific meaning of the above terms in the context of the present invention may be understood in specific cases.

### Embodiment

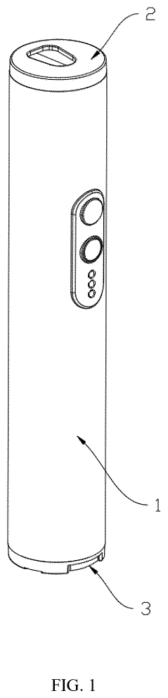

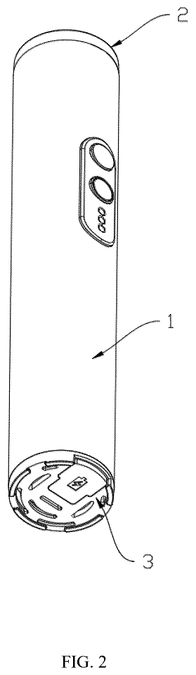

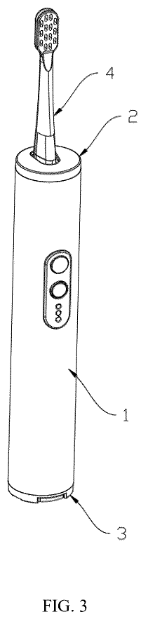

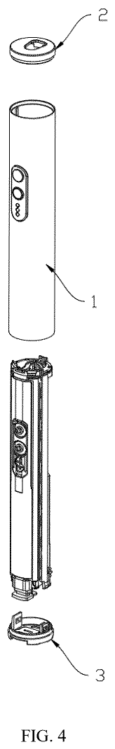

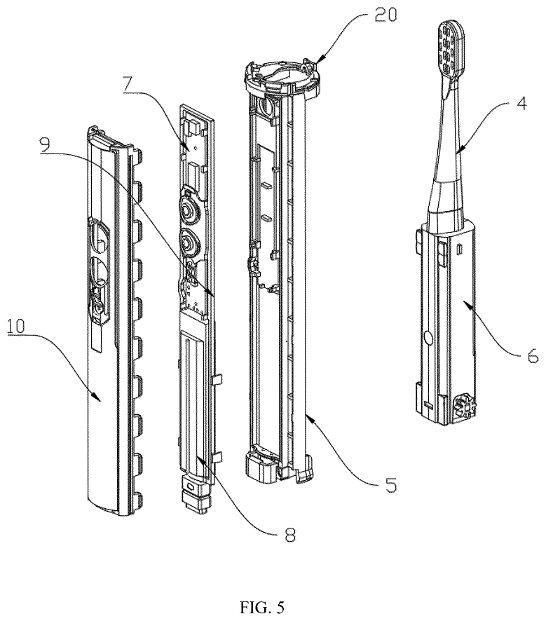

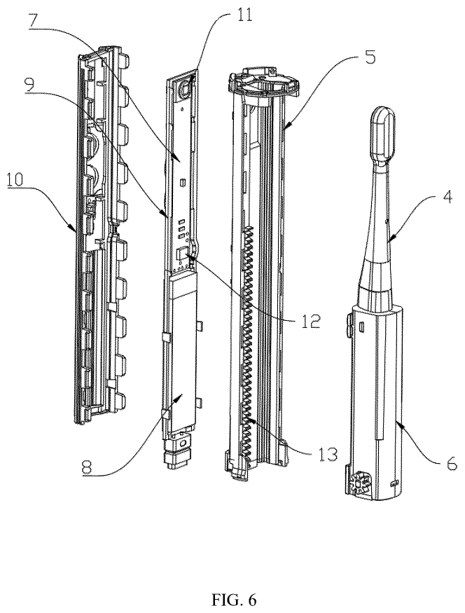

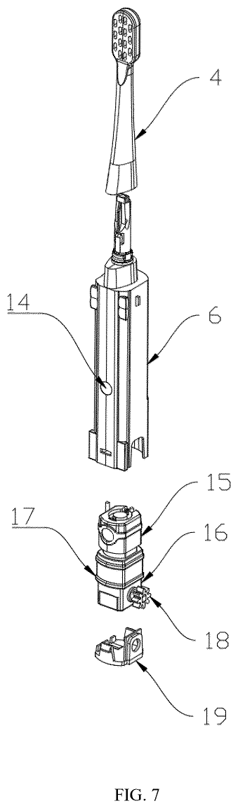

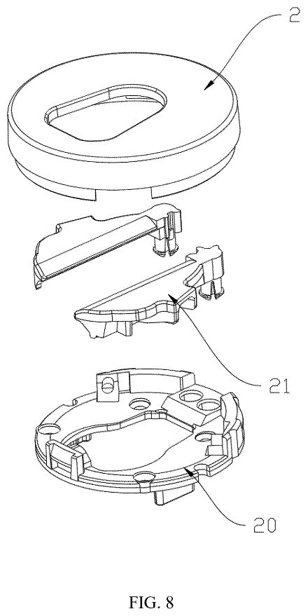

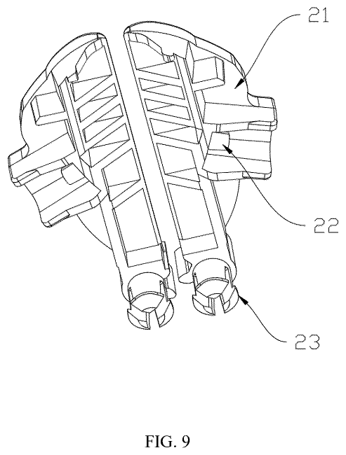

As shown from FIG. 1 to FIG. 9:

The invention provides a retractable electric toothbrush, comprising a handle 1 and a toothbrush head 4; a mounting plate 5 is fixedly installed inside the handle 1; one side of the mounting plate 5 is slidably connected to a mounting cylinder 6, and a driving assembly is arranged between the mounting cylinder 6 and the mounting plate 5; the driving assembly is configured to drive the mounting cylinder 6 to move vertically inside the handle 1, thereby causing the toothbrush head 4 to extend or retract into the handle 1; a control mainboard 7 is installed on an other side of the mounting plate 5, and a waterproof cover plate 10 is connected to an outer side adjacent to the control mainboard 7; a sealing gasket 9 is connected between the waterproof cover plate 10 and the mounting plate 5, ensuring waterproof performance of an electrical-control component on one side of the mounting plate during use, thereby guaranteeing long-term stable operation.
Wherein, an electric motor core for driving the toothbrush head 4 is installed at a top end of the mounting cylinder 6, and the toothbrush head 4 is detachably connected to a driving end of the electric motor core, facilitating periodic disassembly and replacement of the toothbrush head 4.
Wherein, the driving assembly comprises a rack 13 fixedly installed on one side of the mounting plate 5 and a drive motor 15 fixedly installed inside the mounting cylinder 6; an output end of the drive motor 15 is transmission-connected to a speed reducer 16; a drive shaft is provided on one side of the speed reducer 16, and a gear 18 is fixedly sleeved at an end of the drive shaft, the gear 18 meshes with the rack 13 to drive the mounting cylinder 6 to move vertically rapidly, thereby achieving extension and retraction of the toothbrush head 4.
Wherein, a sealing ring 17 is fixedly sleeved on an outer side of the speed reducer 16, and the sealing ring 17 is sealingly connected to an inner wall of the mounting cylinder 6, ensuring a well-sealed space for the drive motor 15 to prevent water ingress, thereby guaranteeing overall waterproof performance; a locking base 19 is connected to a bottom end of the speed reducer 16, and the locking base 19 is tightly clamped to a bottom end of the mounting cylinder 6 to ensure stable installation of the speed reducer 16.
Wherein, both left and right ends on one side of the mounting plate 5 are provided with limit sliding grooves, and both left and right ends on one side of the mounting cylinder 6 are provided with limit strips; the limit strips are slidably installed in the corresponding limit sliding grooves to ensure high stability and smoothness during movement adjustment of the mounting cylinder 6.
Wherein, a battery 8, a disinfection component 11, and a Hall element 12 are fixedly installed on the control mainboard 7. When the toothbrush head 4 is fully retracted inward, the disinfection component 11 is provided corresponding to the toothbrush head 4; a through slot is provided on the mounting plate 5 near the installation position of the disinfection component 11, and an end of the disinfection component 11 extends through the through slot; waterproof sealant is filled between the through slot and the end of the disinfection component 11 to ensure sealing and prevent water leakage. The disinfection component 11 is an ultraviolet disinfection lamp.
Wherein, a magnet 14 is fixedly embedded on one side of the mounting cylinder 6. When the toothbrush head 4 is fully extended outward, the magnet 14 is provided corresponding to the Hall element 12 to enable rapid detection of the position of the mounting cylinder 6, allowing immediate stoppage upon reaching the designated position.
Wherein, an upper cover 2 and a lower cover 3 are fixedly installed at upper and lower ends of the handle 1, respectively; a center of the upper cover 2 is provided with a through hole for the toothbrush head 4 to extend out; the lower cover 3 is provided with a plurality of drainage holes and a charging port; a waterproof cover is connected to the charging port, and a charging connector is fixedly installed inside the charging port, with the charging connector electrically connected to the control mainboard 7.
Wherein, two baffles 21 are installed on a bottom of the upper cover 2 to seal the through hole of the upper cover 2, ensuring rapid light blocking during sterilization by the disinfection component 11 for enhanced safety during use; a fixed disk 20 is fixedly installed at a top end of the mounting plate 5, and the fixed disk 20 is provided with a through slot corresponding to the through hole; two sets of connection holes are provided at one end of the fixed disk 20 near the through slot; one end of each baffle 21 is provided with a mounting shaft 23, and the mounting shafts 23 are rotatably installed in the corresponding connection holes; fixed shafts are provided on both sides of the fixed disk 20 near the through slot, and connection shafts 22 corresponding to the fixed shafts are provided on one side of each baffle 21; springs (not shown in the drawings) are connected between the corresponding connection shafts 22 and the fixed shafts.
Wherein, one end of each baffle 21 near the mounting shaft 23 is shaped as an arc, and arc surfaces are provided with gear slots; the gear slots of two baffles 21 mesh with each other to ensure synchronous movement of two baffles 21, achieving synchronized opening and closing; a bottom side of each baffle 21 is provided with an inclined surface; when the toothbrush head 4 extends outward, its end drives the baffles 21 to compress the springs by pressing against inclined surfaces, thereby completing the opening of the baffles 21.
Working principle of the embodiment: the invention achieves rapid movement of the toothbrush head 4 during use through the mounting plate 5 and driving assembly installed inside the handle 1, enabling quick retractable adjustment, which facilitates swift storage and protection of the toothbrush head 4 during use or transportation, preventing contamination or damage. Additionally, the disinfection component 11 installed inside the handle 1 provides rapid disinfection when the toothbrush head 4 is retracted, ensuring readiness for subsequent use.

The embodiments of the invention are provided for illustrative and descriptive purposes, and are neither exhaustive nor restrictive to the disclosed form. Numerous modifications and variations will be apparent to those of ordinary skill in the art. The selection and description of embodiments aim to better explain the principles and practical applications of the invention, enabling skilled practitioners to comprehend the invention and design various modified embodiments suitable for specific purposes.

1. A retractable electric toothbrush, comprising a handle and a toothbrush head; a mounting plate is fixedly installed inside the handle; one side of the mounting plate is slidably connected to a mounting cylinder, and a driving assembly is arranged between the mounting cylinder and the mounting plate; the driving assembly is configured to drive the mounting cylinder to move vertically inside the handle, thereby causing the toothbrush head to extend or retract into the handle; a control mainboard is installed on an other side of the mounting plate, and a waterproof cover plate is connected to an outer side adjacent to the control mainboard; a sealing gasket is connected between the waterproof cover plate and the mounting plate.

2. The retractable electric toothbrush of claim 1, wherein an electric motor core for driving the toothbrush head is installed at a top end of the mounting cylinder, and the toothbrush head is detachably connected to a driving end of the electric motor core.

3. The retractable electric toothbrush of claim 1, wherein the driving assembly comprises a rack fixedly installed on one side of the mounting plate and a drive motor fixedly installed inside the mounting cylinder; an output end of the drive motor is transmission-connected to a speed reducer; a drive shaft is provided on one side of the speed reducer, and a gear is fixedly sleeved at an end of the drive shaft, the gear meshes with the rack.

4. The retractable electric toothbrush of claim 3, wherein a sealing ring is fixedly sleeved on an outer side of the speed reducer, and the sealing ring is sealingly connected to an inner wall of the mounting cylinder; a locking base is connected to a bottom end of the speed reducer, and the locking base is tightly clamped to a bottom end of the mounting cylinder.

5. The retractable electric toothbrush of claim 1, wherein both left and right ends on one side of the mounting plate are provided with limit sliding grooves, and both left and right ends on one side of the mounting cylinder are provided with limit strips; the limit strips are slidably installed in the corresponding limit sliding grooves.

6. The retractable electric toothbrush of claim 1, wherein a battery, a disinfection component, and a Hall element are fixedly installed on the control mainboard; when the toothbrush head is fully retracted inward, the disinfection component is provided corresponding to the toothbrush head; a through slot is provided on the mounting plate near the installation position of the disinfection component, and an end of the disinfection component extends through the through slot; waterproof sealant is filled between the through slot and the end of the disinfection component; the disinfection component is an ultraviolet disinfection lamp.

7. The retractable electric toothbrush of claim 6, wherein a magnet is fixedly embedded on one side of the mounting cylinder; when the toothbrush head is fully extended outward, the magnet is provided corresponding to the Hall element.

8. The retractable electric toothbrush of claim 1, wherein an upper cover and a lower cover are fixedly installed at upper and lower ends of the handle, respectively; a center of the upper cover is provided with a through hole for the toothbrush head to extend out; the lower cover is provided with a plurality of drainage holes and a charging port; a waterproof cover is connected to the charging port, and a charging connector is fixedly installed inside the charging port, with the charging connector electrically connected to the control mainboard.

9. The retractable electric toothbrush of claim 8, wherein two baffles are installed on a bottom of the upper cover to seal the through hole of the upper cover; a fixed disk is fixedly installed at a top end of the mounting plate, and the fixed disk is provided with a through slot corresponding to the through hole; two sets of connection holes are provided at one end of the fixed disk near the through slot; one end of each baffle is provided with a mounting shaft, and the mounting shafts are rotatably installed in the corresponding connection holes; fixed shafts are provided on both sides of the fixed disk near the through slot, and connection shafts corresponding to the fixed shafts are provided on one side of each baffle; springs are connected between the corresponding connection shafts and the fixed shafts.

10. The retractable electric toothbrush of claim 9, wherein one end of each baffle near the mounting shaft is shaped as an arc, and arc surfaces are provided with gear slots; the gear slots of two baffles mesh with each other to ensure synchronous movement of two baffles, achieving synchronized opening and closing; a bottom side of each baffle is provided with an inclined surface; when the toothbrush head extends outward, its end drives the baffles to compress the springs by pressing against inclined surfaces, thereby completing the opening of the baffles.
